(19) United States
(12) Patent Application Publication
(10) Pub. No.: US 2013/0274577 A1
(43) Pub. Date: Oct. 17, 2013

(54) Medication Delivery Device and Related Methods of Use

(71) Applicants: Farid AMIROUCHE, Highland Park, IL (US); Matthew L. Cantwell, Northbrook, IL (US)

(72) Inventors: Farid AMIROUCHE, Highland Park, IL (US); Matthew L. Cantwell, Northbrook, IL (US)

(73) Assignee: PicoLife Technologies

(21) Appl. No.: 13/470,140

(22) Filed: May 11, 2012

Related U.S. Application Data
(63) Continuation-in-part of application No. 13/448,013, filed on Apr. 16, 2012.

(51) Int. Cl. A61M 5/168; A61B 5/145; A61M 5/142

(52) U.S. Cl. 600/365; 604/151; 604/66

(57) ABSTRACT

Embodiments of the disclosure relate to medical devices and, in particular, to devices for delivery of basal and/or bolus doses of one or more medicaments to a patient. In one embodiment, a medical device for delivering a medicament to the body of a user may include a reservoir configured to contain the medicament, a delivery mechanism for channeling the medicament from the reservoir to the user, a pumping mechanism for pumping the medicament from the reservoir, through the delivery mechanism, and to the user, wherein the medical device may be configured to deliver a dose of the medicament to the user. The medical device may also include a sensor for monitoring a parameter of the body and a display of information to alert and keep the user informed.

### CROSS-REFERENCE TO RELATED APPLICATIONS

This application is a continuation in part of U.S. application Ser. No. 13/448,013, filed on Apr. 16, 2012, the entirety of which is incorporated herein by reference.

### FIELD OF THE DISCLOSURE

Embodiments of the present disclosure relate to the field of medical devices and, in particular, to devices for delivery of basal and/or bolus doses of one or more medicaments to a patient, for example, subcutaneously. More specifically, embodiments of the present disclosure are directed to a medication delivery pump system that utilizes siliconized membrane diffusion and includes an external infusion device, a medication cartridge, and a delivery mechanism, for instance, a button, a switch, a trigger, or a lever.

Embodiments of the present disclosure also relate to devices and methods of using a drug delivery pump having a disposable cartridge system featuring collapsible reservoirs and dynamic membranes that are magnetically driven, an adaptable infusion set, and a continuous glucose monitor.

### BACKGROUND OF THE DISCLOSURE

Diabetes is a complex disease caused by the body's failure to produce adequate insulin or a cell's failure to respond to insulin, resulting in high levels of glucose in the blood. Type I diabetes is a form of diabetes mellitus that results from autoimmune destruction of insulin-producing beta cells of the pancreas in genetically predisposed individuals. There is no current cure, and treatment by injection or infusion of insulin must be continued indefinitely. Type II diabetes is a metabolic disorder brought on at any age by a combination of lifestyle, diet, obesity, and genetic factors. The World Health Organization recently revised its findings from a study conducted in 2004 with predictions that by 2030, 10% of the world's population of all ages will have either Type I or Type II diabetes. This translates to roughly 552 million people worldwide suffering from some form of this disease.

Typically, treatment for diabetes requires both repeated checking of blood glucose levels and several injections of insulin as prescribed by the physician throughout the day, because insulin cannot be taken orally. Major drawbacks of such treatment are the constant need to draw blood and test glucose levels throughout the day, improper or low dosage amounts of insulin, contamination of the insulin delivery system, lifestyle restriction, the unfortunate potential development of subcutaneous scar tissue due to repeated injections at the same location, and the high cost of medication, testing strips, and other treatment-related materials.

Diabetes is usually controlled by insulin replacement therapy in which insulin is delivered to the diabetic person by injection to counteract elevated blood glucose levels. Recent therapies include the basal/bolus method of treatment in which a basal dose of a long-acting insulin medication, such as, for example, Humalog® or Apidra®, is delivered via injection once every day, or, in the alternative, gradually throughout the day. The basal dose provides the body with an insulin profile that is relatively constant throughout the day, or could follow a profile best-suited for the particular diabetic patient. These rates can change based on the patient's response to insulin. At mealtime, an additional dose of insulin, or a bolus dose, may be administered based on the amount of carbohydrate and protein in the meal. The bolus dose is viewed as an emergency response to spikes in blood sugar that need to be brought down or otherwise controlled by injection of insulin. Accurate calculations of various parameters, including, but not limited to, the amount of carbohydrates and proteins consumed, and the lapse in time since the last dosage, are necessary to determine the appropriate dosage of insulin. The dosages are thus prone to human error, and the method is ineffective when doses are skipped, forgotten, or miscalculated. Exercise, stress, and other factors can also cause the calculations to be inaccurate. Bolus doses are usually administered when the patient's glucose level is high or above certain acceptable thresholds and needs immediate attention.

To address these and other problems, insulin delivery devices or pumps were developed to mimic the way a normal, healthy pancreas delivers insulin to the body. Innovations strove to improve diabetic treatment by, for instance, increasing patient compliance with treatment, and helping to decrease the number of hyper- and hypoglycemic events. Ambulatory devices focused mainly on improving portability and discreteness, but these bolus delivery systems have numerous drawbacks. Additionally, while disposable insulin devices have increased in popularity, the cost to the patient of such devices has also increased approximately 62% per year.

One drawback is that these devices often require repeated injections of insulin throughout the day, also known as multiple daily injections (MDI). Further, these devices often require larger amounts of physical force to inject them into the skin. Particularly when combined with the need for multiple injections, this may decrease patient compliance, creating the same issues that the devices were often developed to solve. Such force requirements may also be difficult for those with limited mobility due to age or other restrictions. Thus, it would be highly desirable to decrease the ease of medicament delivery and/or the number of injections required.

Many devices also often have problems with accuracy. For instance, the amount of pressure required to expel the medication from the devices frequently caused the devices to fail. Another drawback is that the accuracy of insulin doses may depend on the accuracy of ratcheting mechanisms inside the device. This accuracy, in turn, depends on tight tolerances and consistent manufacturing standards, which can vary among brands.

Additionally, many devices are refillable. The refilling process may cause bubbles to form inside the medication cartridge that may prove difficult to expel. Even very small bubbles of 10 microliters or less can displace enough fluid to equal a missed dose of 1 unit of medicament. Refilling the device may also increase the likelihood of contamination, and thus infection. Insulin medication itself can also form bubbles when dissolved air is “outgassed” through normal changes in temperature or atmospheric pressure. Therefore, the need exists to provide a more accurate, disposable, prefilled medical device with increased sterility that is also affordable to the consumer.

Another recurring problem with many miniaturized ambulatory infusion pumps is that the amount of medication that can be stored in the reservoirs often cannot meet the needs of certain diabetic patients. Many Type II diabetics who require insulin often need more insulin per gram of carbohydrate due to a condition referred to as “insulin resistance.” Therefore, a substantial need exists to maximize the volume of the medication reservoirs to address the needs of both Type I and Type II diabetic patients by allowing continuous subcutaneous insulin infusion therapy while maintaining a very small overall size of the device itself.

### SUMMARY OF THE DISCLOSURE

Embodiments of the present disclosure relate to the field of healthcare medical devices and, in particular, to devices for medicament delivery and continuous monitoring.

In one embodiment, a medical device for delivering a medicament to the body of a user may include a reservoir configured to contain the medicament, a delivery mechanism for channeling the medicament from the reservoir to the user, a pumping mechanism for pumping the medicament from the reservoir, through the delivery mechanism, and to the user, wherein the medical device is configured to deliver a basal dose of the medicament to the user. The medical device may also include a first sensor for monitoring a parameter of the body.

Various embodiments of the medical device may include one or more of the following features: the elongate housing may include a securing mechanism configured to secure the elongate housing to an article of clothing; the elongate housing may include dimensions allowing the housing to be disposed substantially within a pocket of the article of clothing; the reservoir may be configured to contain a plurality of medicaments; the pumping mechanism may be configured to deliver a first medicament of the plurality of medicaments at a first rate and deliver a second medicament of the plurality of medicaments at a second rate, wherein the second rate is different from the first rate; the pumping mechanism may be configured to selectively deliver bolus doses to the user; the housing may include an actuator configured to allow the user to instruct the medical device to deliver a bolus dose of the medicament; the elongate housing may include a graphical display screen; the first sensor may be configured to continuously monitor the parameter; the parameter may be blood glucose; the first sensor may include a portion in communication with blood of the user; the portion may be implanted within the body of the user; the portion may be wirelessly coupled to the first sensor; the device may further comprise a second sensor analyzing a blood sample removed from the body for the parameter; the reservoir may be selectively removable from the housing; the pumping mechanism may include a magnet and an electromagnetic coil; the housing may include electronics for controlling the first sensor and the pumping mechanism; the device may further comprise a handheld controller in wireless communication with the electronics; the handheld controller may include a second sensor configured to analyze a blood sample removed from the body for the parameter; the elongate housing may include a length in the range of about 3.5 inches to 4.5 inches, and a width in the range of about 0.40 inches to 0.6 inches; the elongate housing may include a substantially cylindrical configuration; and the elongate housing may be substantially pen-shaped.

In another embodiment, a pump mechanism for delivering a medicament from a reservoir, wherein the reservoir is removably secured to a housing, may include a first elongate pump insert body having a first opening configured to receive a first magnet, a second elongate pump insert body having a second opening configured to receive a second magnet, a flexible membrane disposed in between the first and second elongate pump insert bodies, and a plurality of electromagnetic coils.

Various embodiments of the pump mechanism may include one or more of the following features: the first and second magnets may be disposed in the first and second openings; the flexible membrane may be operably coupled to one of the first and second magnets; the reservoir may be disposed in a housing configured to be secured to an article of clothing.

In a further embodiment, a medical device for delivering a medicament to the body of a user may include an elongate housing, a reservoir configured to contain the medicament, a delivery mechanism for channeling the medicament from the reservoir to the user, a pumping mechanism for pumping the medicament from the reservoir and through the delivery mechanism to the user. The pumping mechanism may include a first elongate pump insert body having a first opening configured to receive a first magnet, a second elongate pump insert body having a second opening configured to receive a second magnet, a flexible membrane disposed in between the first and second elongate pump insert bodies, and a plurality of electromagnetic coils. The medical device may further include a first sensor for monitoring a parameter of the body, wherein the medical device is configured to deliver a basal dose of the medicament to the user.

Moreover, those skilled in the art will appreciate that the conception upon which this disclosure is based may readily be used as a basis for designing other structures, methods, and systems for carrying out the several purposes of the present disclosure. It is important, therefore, to recognize that the claims should be regarded as including such equivalent constructions insofar as they do not depart from the spirit and scope of the present disclosure.

### BRIEF DESCRIPTION OF THE DRAWINGS

The accompanying drawings illustrate certain exemplary embodiments of the present disclosure, and together with the description, serve to explain principles of the present disclosure.

FIG. 1A depicts a perspective view of an exemplary miniature medicament delivery device and continuous monitoring system, in accordance with an embodiment of the present disclosure;

FIGS. 1B-1C depict exploded views of the exemplary miniature medicament delivery device and continuous monitoring system of FIG. 1A;

FIG. 2 illustrates an exemplary cartridge system for use with the exemplary miniature medicament delivery and continuous monitoring system of FIGS. 1A-1C, in accordance with an embodiment of the present disclosure;

FIG. 3A depicts an exemplary clamshell assembly for use with the exemplary miniature medicament delivery and continuous monitoring system of FIGS. 1A-1C, in accordance with an embodiment of the present disclosure;

FIG. 3B depicts a bottom view of the exemplary clamshell assembly of FIG. 3A;

FIG. 3C depicts a side view of the exemplary clamshell assembly of FIGS. 3A-3B;

FIG. 4A illustrates an exemplary pump component for use with the exemplary miniature medicament delivery and continuous monitoring system of FIGS. 1A-1C, in accordance with an embodiment of the present disclosure;

FIG. 4B illustrates a bottom view of the exemplary pump component of FIG. 4A;

FIGS. 5A-5D depict various views of an exemplary pump component for use with the exemplary miniature medicament delivery and continuous monitoring system of FIGS. 1A-1C, in accordance with an embodiment of the present disclosure;

FIGS. 6A-6B depict various views of an exemplary inlet/outlet member for use with the exemplary miniature medicament delivery and continuous monitoring system of FIGS. 1A-1C, in accordance with an embodiment of the present disclosure;

FIGS. 7A-7C depict various views of a reservoir housing for use with the exemplary miniature medicament delivery and continuous monitoring system of FIGS. 1A-1C, in accordance with an embodiment of the present disclosure;

FIG. 8 depicts a membrane for use with the exemplary miniature medicament delivery and continuous monitoring system of FIGS. 1A-1C, in accordance with an embodiment of the present disclosure;

FIG. 9 shows a perspective view of an electromagnetic coil for use with the exemplary miniature medicament delivery and continuous monitoring system of FIGS. 1A-1C, in accordance with an embodiment of the present disclosure;

FIGS. 10A-10B depict perspective views of an exemplary miniature medicament delivery device and continuous monitoring system, in accordance with another embodiment of the present disclosure;

FIG. 11 is an exploded view of the exemplary miniature medicament delivery device and continuous monitoring system of FIGS. 10A-10B;

FIGS. 12A-12B depict perspective views of upper and lower housings for use with the exemplary miniature medicament delivery and continuous monitoring system of FIGS. 10A-10B, in accordance with an embodiment of the present disclosure;

FIG. 13 illustrates an exemplary cartridge system for use with the exemplary miniature medicament delivery and continuous monitoring system of FIGS. 10A-10B, in accordance with an embodiment of the present disclosure;

FIG. 14 is an exploded view of the exemplary cartridge system of FIG. 13;

FIGS. 15A-15B depict perspective views of various pump components for use with the exemplary miniature medicament delivery and continuous monitoring system of FIGS. 10A-10B, in accordance with an embodiment of the present disclosure;

FIG. 16 illustrates an exemplary reservoir housing for use with the exemplary miniature medicament delivery and continuous monitoring system of FIGS. 10A-10B, in accordance with an embodiment of the present disclosure;

FIGS. 17A-17B depict perspective views of an exemplary inlet/outlet member for use with the exemplary miniature medicament delivery and continuous monitoring system of FIGS. 10A-10B, in accordance with an embodiment of the present disclosure;

FIG. 18 depicts a perspective view of an exemplary cartridge housing for use with the exemplary miniature medicament delivery and continuous monitoring system of FIGS. 10A-10B, in accordance with an embodiment of the present disclosure; and

FIG. 19 illustrates an exploded view of an exemplary clamshell assembly for use with the exemplary miniature medicament delivery and continuous monitoring system of FIGS. 10A-10B, in accordance with an embodiment of the present disclosure.

### DETAILED DESCRIPTION OF THE PREFERRED EMBODIMENTS

Reference will now be made in detail to the exemplary embodiments of the present disclosure described below and illustrated in the accompanying drawings. Wherever possible, the same reference numbers will be used throughout the drawings to refer to same or like parts.

While the present invention is described herein with reference to illustrative embodiments for particular applications, it should be understood that the invention is not limited thereto. Those having ordinary skill in the art and access to the teachings provided herein will recognize additional modifications, applications, embodiments, and substitution of equivalents all fall within the scope of the invention. Accordingly, the invention is not to be considered as limited by the foregoing or following descriptions.

Other features and advantages and potential uses of the present disclosure will become apparent to someone skilled in the art from the following description of the disclosure, which refers to the accompanying drawings.

Prior to providing a detailed description of the embodiments disclosed herein, however, the following overview is provided to generally describe the contemplated embodiments. Further, although the embodiments disclosed herein are described in connection with monitoring blood glucose, those of ordinary skill in the art will understand that the principles of the present disclosure may be suitable for monitoring any body parameter, including, e.g., blood pressure, cholesterol levels, sodium levels, medicament saturation levels, and so forth. Further, although the embodiments disclosed herein are described in connection with delivery of, e.g., insulin to treat diabetes, those of ordinary skill in the art will understand that any suitable therapeutic agent may be delivered to a patient, regardless of whether the agent is delivered to treat a disease state. For example, the embodiments disclosed herein may deliver medicaments for pain management or joint lubrication, or may be used for reverse controlled fluid extraction.

The disclosed embodiments relate to a miniature medicament delivery and, among other things, continuous glucose monitoring device. The term “fluid” may include a state of matter or substance (liquid or gas), whose particles can move about freely and have no fixed shape, but rather conform to the shape of their containers. Further, the term “channel” may include a passage for fluids to flow through. Moreover, the term “medicament” may be used to refer to a substance used in therapy, a substance that treats, prevents, or alleviates the symptoms of disease, a medicine in a specified formulation, an agent that promotes recovery from injury or ailment, or any other fluid used in the treatment or diagnosis of a patient.

Embodiments of the disclosure described herein overcome at least certain disadvantages of the prior art by providing a drug delivery infusion device with a cartridge system featuring a reservoir. This reservoir may be collapsible. The reservoir may be integrated to be actuated either manually or through automatic means, for instance, through the use of a programmable controller. The cartridge system may be prefilled and disposable. In other embodiments, the cartridge system may be refilled by any suitable means.

Embodiments of the present disclosure rely on data obtained from a continuous glucose metering and sensor device fully or partially imbedded in the user's body, and/or in conjunction with a manual test strip reader to determine the basal and bolus insulin levels of the user. Exemplary continuous glucose metering and sensor devices are described in U.S. patent application Ser. No. 13/448,013, filed on Apr. 16, 2012, the entirety of which is incorporated herein by reference. In some instances, embodiments of the present disclosure may be configured to receive data that is obtained by a separate sensing device and then automatically or manually entered into the drug-delivery device or any associated component thereof. This data may then become part of the algorithm that automatically delivers the desired bolus amount of medication into the user's body. The device may calculate the user's blood glucose level, and the result may be displayed on the display of the device. In addition, any suitable means of communicating the user's blood glucose level to the user may be employed. Such means may include, but is not limited to, e.g., an audible announcement of calculated glucose level, a vibratory indication, and/or a tactile indication.

If the glucose level is within range, then no action by the device may be needed. If the glucose level is too high, or above the prescribed threshold, a bolus dose of insulin can be administered either by the user manually, for instance, with the depression of the delivery button, or the device can be programmed to do this automatically. Further, the prescribed threshold may be variable. In addition to this function, a complete history of basal corrections and bolus delivery may be stored in the device for use by the patient or by a healthcare provider for assessment and monitoring of the patient's healthcare. The stored history may be communicated, e.g., wirelessly, to a central database or the healthcare provider for evaluation. Evaluation may occur either with the patient directly, for instance the data may be downloaded during a patient visit, or remotely, for instance, transmitted to a database on an ongoing basis.

More specifically, embodiments of the present disclosure may include a cartridge system having one or more collapsible reservoirs with a volume, preferably, of approximately 0.5 ml to 3.0 ml, with a more preferred volume of 1.2 ml. The one or more reservoirs may be elastomeric and may have properties that allow the reservoirs to conform to the volume requirements of the medicaments. For instance, the reservoir(s) may include molded polydimethylsiloxane (PDMS) or other suitable materials. If more than one reservoir is present, each reservoir may interconnect in any combination to serve as complement to one another. In such an embodiment, the reservoirs can be filled with a single medicament, or may be filled with a mixture of different or similar medicaments. Further, embodiments having a plurality of reservoirs may aid in reconstituting one or more medicaments. For example, one reservoir may contain a powdered form of a medicament, while another reservoir contains a suitable liquid, e.g., water or a saline solution. Some medicaments have comparatively short shelf-lives in liquid form, so it would be desirable to store a powdered form in the drug delivery device and reconstitute the medicament to a liquid form just prior to injecting the medicament into the patient. If only a single reservoir is present, the single reservoir may be filled with either one medicament, or a plurality of different medicaments. In one embodiment, the reservoir can be pre-filled with insulin.

The basic mechanism of the drug delivery device is to actuate the fluid chamber and membrane magnetically. The membrane of the actuation chamber is placed between two suitable magnets, such as, e.g., gold-plated neodymium-iron-boron disk magnets that may be housed within the pump body insert. The pump body cartridge insert may have a fluid receiving opening, a fluid discharge opening, an inlet channel and an outlet channel. The pump body insert may be placed between the inlet/outlet members. The inlet/outlet members may have a fluid receiving opening, a fluid discharge opening, and a fluid outlet component. Additionally, the inlet/outlet members may include a male part that securely engages to a female part of the reservoir forming an airtight seal. The reservoir housing may include a gripping lid to fix the inlet/outlet channels of the cartridge unit to an inlet of the reservoir. The reservoir, the fluid receiving opening of the inlet/outlet member, the fluid receiving opening, the inlet channel, the outlet channel, and the fluid discharge opening of the pump body insert, the fluid discharge opening and the fluid outlet component of the inlet/outlet member may be in fluid communication. The cartridge system may further include valve membranes that are placed between the fluid receiving opening of the pump body insert and the inlet/outlet members, and between the fluid discharge opening of the pump body insert and the inlet/outlet members.

The valve membranes of the cartridge system can be active valves magnetically operated and integrated into the membrane housing to control the opening and closing of the output flow. A feedback control may be utilized to allow for automatic opening or closing of the valve and dispersion of the medicament associated with the reservoir and the corresponding valve. In other embodiments, opening and closing of the valve and dispersion of the medicament may be manually controlled.

The present disclosure may also include a cartridge system having one or more orifices to fill or refill a medicament or multiple medicaments in the reservoir. The one or more orifices can be located on the reservoir, or on the inlet/outlet members, and the one or more orifices may be in fluid communication with the reservoir.

The present disclosure may further include a method of delivering medicament using a drug delivery device having a cartridge system with a collapsible inner chamber. The method may include the steps of providing a drug delivery device having a pump driver system and a cartridge system, such as, e.g., a planar cartridge system, loading a prefilled reservoir containing fluid medicament into the cartridge system, engaging securely with the cartridge system and the pump driver system, selecting various parameters on a user interface of the pump driver system, including selecting predetermined values or specifying user-defined values for the parameters, and connecting suitable delivery mechanisms, such as, e.g., an infusion set, to the drug delivery device.

The collapsible reservoir may be permanently adhered to the valve covers creating an air-tight seal, and may be separated within the housing by a thin shield, for instance, a polymer shield. The cartridge may be part of a magnetic medication pump, and, hence, may contain the dynamic flow control channels and valves, as well as the magnets that may be part of the design, as discussed below. As a result, the reservoir may remain permanently sterile and impervious to outside contaminants. The medication delivery pump assembly of the present disclosure may be worn outside the body, and the medication may be dispensed into the body via an attachable infusion set, which may be connected to a suitable outlet. Embodiments of the present disclosure may further include an actuation mechanism, for instance, a button, a switch, a lever, a knob, or a trigger, to manually deliver a precise dose of insulin to a patient. Other embodiments of the present disclosure may use a far-field radio frequency communication system to integrate the pump with a control unit, for instance, a hand-held remote control device. Those of ordinary skill will recognize that any suitable wired or wireless (e.g., infrared, Bluetooth, Wi-Fi, etc.) means of communication may be used. The drug delivery system or pump may further include a digital remote controller that wirelessly communicates with the pump control unit, operating and controlling the delivery of the drug through the interface of a cartridge. Further, the control unit may include a data acquisition system, for instance, a program or series of algorithms, configured to store or process data input from the sensor.

The method of delivering medicament using the drug delivery device disclosed herein may include the additional steps of placing an infusion set on a body part of a patient, attaching the infusion set to the patient's body, attaching the infusion set to the pump outlets, and commencing drug delivery from the drug delivering device. Although the embodiments of the present disclosure describe an exemplary infusion set, any suitable mechanism for delivering medicaments to a patient's body may be used.

The method of delivering medicament using the drug delivery device disclosed herein may further include the step of connecting an infusion set to the drug delivery device. The method may also include the steps of connecting one end of one or more catheters having multiple ends, for instance, e.g., one or more Y-shaped catheters, to an outlet component of an inlet/outlet member, and delivering the fluid medicament at a given rate. The step of delivering fluid medicament at a given rate can further include delivering fluid medicament at a controlled and continuous rate for a predetermined or user-defined period of time. Alternatively, the step of delivering fluid medicament at a given rate can further include delivering fluid medicament at a programmable rate that is regulated, e.g., by the patient or by a remote healthcare provider.

The present disclosure also contemplates a method of delivering medicament using the drug delivery device having a cartridge system. The method may include the steps of providing a drug delivery device having a pump driver system and a cartridge system, loading a reservoir to the cartridge system, using an instrument to inject one or more medicaments into the reservoir, engaging the cartridge system securely to the pump driver system, selecting various parameters on a user interface of the pump driver system, including selecting predetermined values or specifying user-defined values for the parameters, and connecting an infusion set to the drug delivery device. The step of connecting an infusion set to the drug delivery device may further include the steps of connecting one end of a Y-shaped catheter to an outlet component of an inlet/outlet member and delivering fluid medicament at a given rate. The step of delivering fluid medicament at a given rate can further include delivering fluid medicament at a controlled and continuous rate for a predetermined or user-defined period of time. Alternatively, the step of delivering fluid medicament at a given rate can further include delivering fluid medicament at a programmable rate that is regulated by the patient. In other embodiments, the rate may vary based on user input or may automatically vary based on preset criteria.

The present disclosure further contemplates a drug delivery device having a pump driver system, a cartridge system, a cannula and an insertion mechanism, and a conduit. The pump driver system may include a driver that drives the magnets that applies forces to the pump membranes of the cartridge system, a controller in communication with the pump to adjust the force applied by the driver, a power source, and a user interface configured to present information to a user. The cartridge system of the device may snap (or otherwise frictionally engage) into the pump drivers of the pump system and is securely engaged to it. The conduit includes a proximal end, a distal end, and a lumen extending from its proximal end to its distal end. The proximal end of the conduit may be securely engaged to the distal end of the cannula and the insertion mechanism, and the distal end may be securely engaged to the proximal end of the fluid outlet component of the inlet/outlet members of the cartridge system.

As alluded to above, embodiments of the present disclosure relate to miniature medicament delivery and continuous monitoring systems, and, more particularly, to miniature insulin electromagnetic micropumps and glucose monitoring systems. The electromagnetic micropumps disclosed herein may be useful for, e.g., delivering insulin to diabetic patients, and also may be used for delivering other drugs to any desired patient. The optionally included continuous glucose monitoring module disclosed here may be useful for determining the level of glucose in a patient's body in a discrete manner. Those of ordinary skill will recognize that the continuous monitoring embodiments disclosed herein may be useful for monitoring any desired body parameter, not just blood glucose.

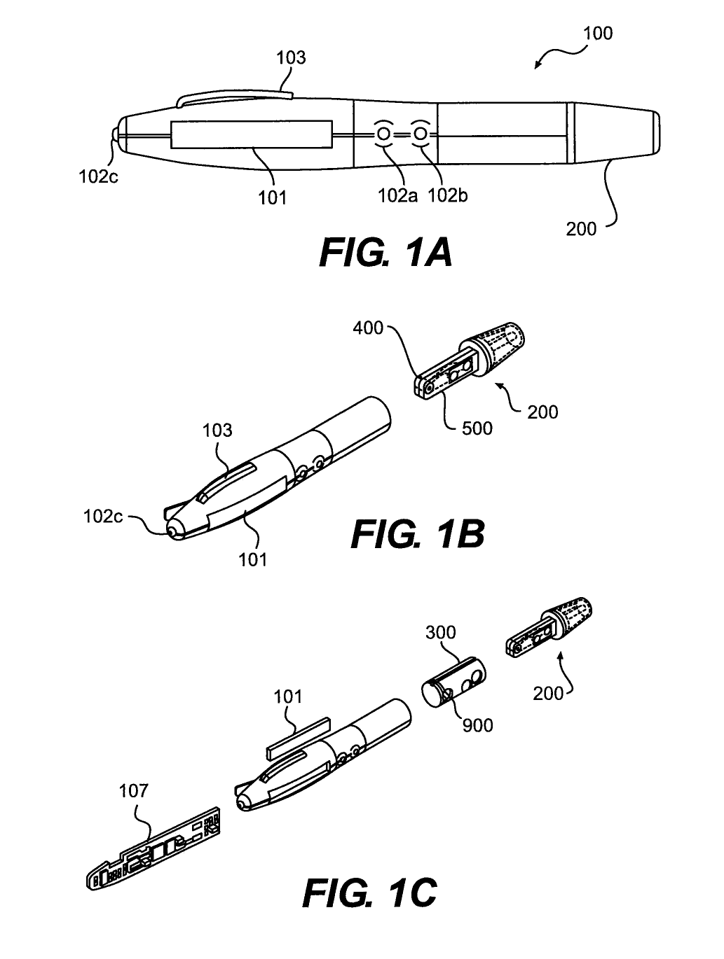

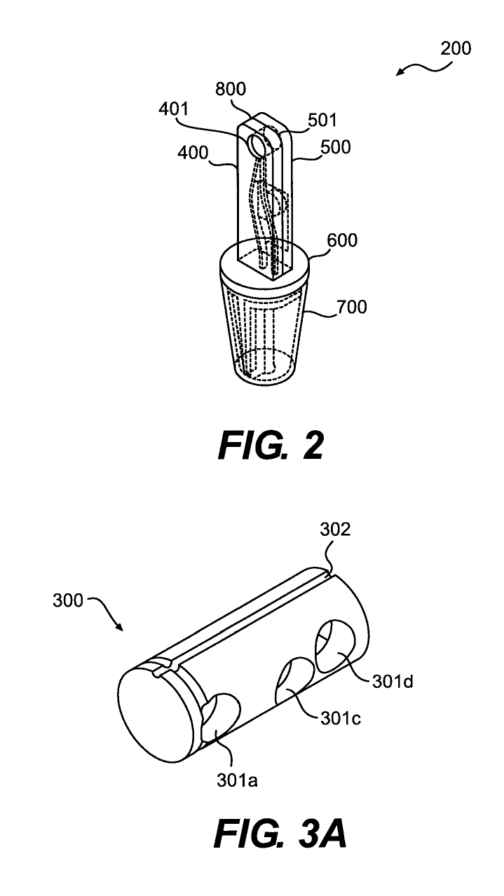

Referring now to the drawings, FIGS. 1A-1C illustrate a medicament delivery device 100, in accordance with an embodiment of the present disclosure. Device 100 may include a display 101, one or more control mechanisms 102, an attachment mechanism 103, and a cartridge system 200. As shown in FIG. 2, cartridge system 200 may further include a plurality of pump insert bodies 400, 500, an inlet/outlet member 600, a reservoir 700, a clamshell 300 having a plurality of coil housings 301, a plurality of magnets (not shown), a plurality of valves (not shown), a guide channel 302, a pump membrane 800, and a plurality of electromagnetic coils 900 (FIG. 9).

Device 100 may be an ‘intelligent’ device that is computer controlled. For instance, device 100 may be programmable, may have a built in glucose monitor device, and may be configured to deliver the right dosage either continuously or at discrete intervals as required. Accordingly, device 100 may be designed to communicate wirelessly with a control device, such as a smart phone, and may be programmed via an application by a healthcare provider or a user.

In one embodiment, device 100 may be configured to be worn close to or directly in contact with the skin. Device 100 may be worn and completely hidden from view, or may be fully or partially visible. In the latter embodiment, device 100 may be configured to look like a common item, allowing it to be visible but still discreet. For instance, as shown in FIG. 1A, device 100 may include a compact, pen-shaped housing. A cartridge may be shaped like a portion of a pen, for instance, a frustum shape for inclusion at an end region of device 100, and may connect to the housing, e.g., by snapping or twisting, to form an integral piece. Pressing an upper button, as a user would a real pen, may allow for manual activation of device 100.

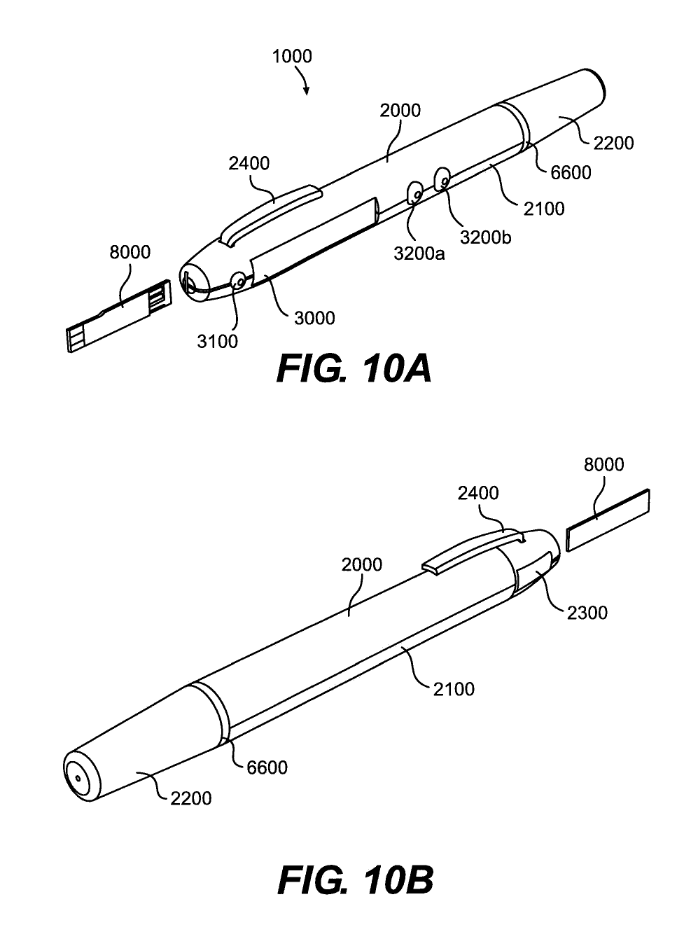

Device 100 may include different colors, designs, or shapes to accommodate the user's preference. For example, users may engrave device 100 with their name or initials, as they might a real pen. Alternatively, device 100 may include designs or a trademark, for example, PicoLife®. A pen-shaped housing may come in a variety of shapes, for instance, the example in FIG. 1A has a wider circumference in a middle region than it does towards the end regions. In other embodiments, such as, e.g., shown in FIGS. 10A-10B, the disclosed device may have a substantially uniform circumference along its length.

Device 100 may also be available in a variety of housing sizes to accommodate a variety of reservoir sizes, for instance, reservoirs ranging from approximately 100 microliters to 350 microliters or less. In one embodiment, the housing of device 100 may be interchangeable, allowing for the same device to be capable of accepting different sized or shaped housings. This may allow a user to alter device 100 according to the size of medicament dosages needed or the length of time over which the user may plan to use device 100. In other embodiments, parts or all of device 100 may be shaped or configured to work with other infusion sets and cannula body interfaces so it can be used as an alternate embodiment for current devices. Further, to facilitate practical, everyday use, device 100 may be lightweight and/or waterproof.

The disclosed device may have any suitable configuration desired. For example, as shown in FIG. 1A, the system may have an elongated, substantially cylindrical configuration, which does not include any sharp edges, thereby allowing the system to be worn or carried by a user, for instance, hidden discretely in, beneath, or attached to a patient's clothing (e.g., a shirt or pants). For example, device 100 may be carried in a pocket of an article of clothing, worn around the patient's neck, or worn on an arm or leg band. Further, while the disclosed embodiment portrays device 100 as having the appearance of a pen, other embodiments of device 100 may take the form or appearance of other items, such as jewelry or accessories, such as a brooch or a watch, for example.

Regardless of the specific shape or configuration chosen, the system of the present disclosure may have a substantially low-profile (e.g., slim profile) configuration. That is, the width or overall bulk of the disclosed device 100 may be selected to allow a user to wear the device 100 close to the skin and discreetly in, on, or beneath clothing. For instance, device 100 may be dimensioned so as to fit within a pocket. In some embodiments, the width of device 100 may be in the range of approximately 0.25 inches to 1.5 inches.

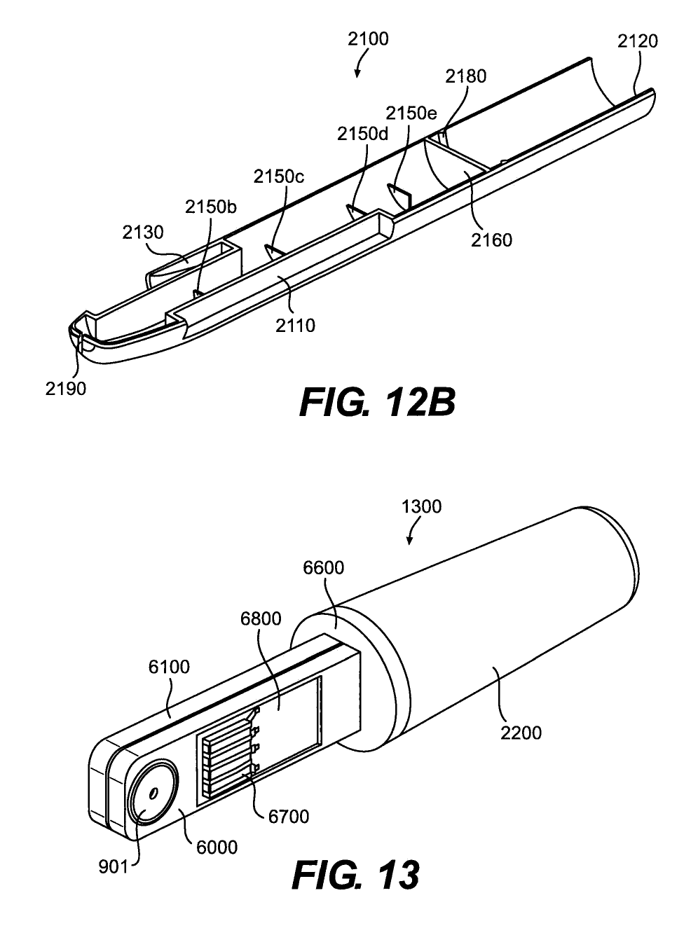

As noted above, device 100 may include a housing. As is shown in FIGS. 12A and 12B, device 100 may include an upper housing 200 and a lower housing 210. Upper and lower housings 200, 210 may be fabricated from any suitable process known in the art. For example, housings 200, 210 may be made from extrusion or molding. Further, housings 200, 210 may be made from any suitable materials. Such materials may include, but are not limited to, polymers, plastics, thermoplastics, and/or elastomers. One suitable material may be Acrylonitrile butadiene styrene (ABS) or equivalents. Housings 200, 210 may be provided with any suitable coating desired. For example, housings 200, 210 may be coated with, e.g., hypoallergenic agents to reduce discomfort to a patient's skin. Further, one or more of housings 200, 210 may be provided with a fragrant coating that may please or soothe a user. Instead of a coating, such agents may be impregnated within one or more external walls of housings 200, 210. Furthermore, housings 200, 210 may be provided in any suitable color or color combination. The housings 200, 210 may be configured to form a hermetic seal when operably coupled. In addition, as will be discussed in greater detail below, housings 200, 210 may be provided with one or more storage locations to store, e.g., blood glucose test strips. Such storage locations may be secured or unsecured. Moreover, cartridge system 220, discussed in greater below, may be of a different color than one or both of housings 200, 210, and cartridge system 220 may be capable of illumination to indicate use or dispensing of medicaments contained therein. In some embodiments, cartridge 210 may be configured so that a user can see the amount of medicament remaining in the cartridge. For instance, the cartridge may be formed of a clear or opaque material.

In some embodiments, the housing and the internal components may be formed of polymer materials. For instance, polymers may be used to make pump insert bodies 400, 500, inlet/outlet member 600, and/or reservoir housings 700. Further, the components of device 100 may be made in any suitable size and through any suitable manufacturing process. To facilitate close proximity with the body, device 100 may be formed and/or manufactured with biocompatible materials. The methods used in the manufacture of the polymer components, as well as the arrangement and design of the cartridge system, may be adapted to commonly used sterilization techniques, such as, e.g., gamma irradiation, steam sterilization, or fluid chemical sterilization.

With specific reference to FIGS. 1A-1C, drug delivery device 100 may include a display 101. Display 101 may be configured to display one or more system parameters (e.g., battery life, error messages), the current time and date, monitored body parameters, date and time of last dose, and any other information that may be communicated to a user of device 100. This information may be conveyed using written words, symbols, pictures, colors, or any other suitable visual means of communication.

Display 101 may be any suitable screen known in the art. For example, display 101 may be a Liquid Crystal Display (LCD) or an Organic Light Emitting Diode (OLED) display capable of black-and-white or colored graphics. Further, display 101 may be configured as a “touch-screen,” permitting a user to control one or more functions of device 100 by touching various portions of the display. In this manner, the user may operate or program device 100 by accessing one or more menus through direct interaction with display 101. Display 101 may be provided a cover or with any suitable coating. Such coatings may protect display 101 from, e.g., scratches or other damage. In addition, the applied coatings may reduce glare, permitting a user to effectively view the contents of display 101 in daylight during outdoor activities. Further, display 101 may be provided with a privacy coating, permitting view of the screen contents at only a predetermined angle of viewing.

A user or operator of device 100 may also control device 100 via one or more control mechanisms 102, such as, e.g., buttons 102a, 102b, and 102c. Buttons 102a, 102b, and 102c may be any suitable button or actuator interface known in the art. For example, buttons 102a, 102b, and 102c may include push buttons, slide buttons, and/or touch buttons that may be activated without any relative movement between the buttons and associated housing. Control mechanisms 102 may alternatively include levers, triggers, switches, knobs, or any other suitable mechanism. Control mechanisms 102 may be located on any suitable surface of a housing (e.g., upper and/or lower housings 200, 210) of device 100. Although the depicted embodiment includes three buttons, those of ordinary skill in the art will recognize that any suitable number of control mechanisms 102 may be included. Further, control mechanisms 102 may be disposed separately from one another or grouped together as desired.

Control mechanisms 102 may be configured to control one or more behaviors of device 100, e.g., on or off, commence or stop medicament delivery, initiate a measurement, or any other suitable behavior. One or more control mechanisms 102 may be multifunctional. That is, a single control mechanism 102 may be capable of executing a plurality of functions. For example, various portions of the same control mechanism may be configured to execute differing functions. In addition, actuating a control mechanism for a short time may execute a first function, while actuating and continuing to actuate the same control mechanism in the same location may be execute a second function.

With specific reference to FIG. 1A, one of buttons 102a, 102b, and 102c may be a bolus button. The bolus button may be linked with suitable electronics and/or mechanical elements to effect delivery of a bolus drug dose. Further, one of buttons 102a, 102b, and 102c may be a multifunctional button. Particularly, one of buttons 102a, 102b, and 102c may allow a user to navigate one or more menus or select between a plurality of options to effectively operate device 100. As such, one of buttons 102a, 102b, and 102c may be capable of multiple functions by, e.g., depressing a first portion of a button to perform a first function and depressing a second portion of the same button to perform a second function. Further, device 100 may be provided with a “home” button, which may be configured to allow a user to clear a selection or to return to a main menu. The home button may also be configured to power on and off device 100, by, e.g., pressing and holding the home button.

Device 100 may also include an attachment mechanism, such as a clip 103, to facilitate portability or storage of device 100. For instance, clip 103 may allow a user to secure device 100 to an item of clothing or an accessory. Any suitable attachment mechanism may be used, for instance, a pin, a clamp, a hook, magnets, hook-and-loop fasteners (Velcro®), or an adhesive.

Device 100 may also include at least one outlet mechanism for delivering medicaments to a patient. In the present disclosure, the outlet mechanism is exemplified by a conventional infusion set known in the art. However, any suitable outlet mechanism may be used. The described infusion set may include a catheter (not shown) having a proximal end, a distal end, and a suitable length therebetween. A proximal end of the catheter may be operably coupled to device 100 and a reservoir therein, and a distal end of the catheter may be operably coupled to a thin, flexible needle suitable for long-term placement into a patient's skin. The catheter may have any suitable configuration and shape, and may be flexible to permit relief of stresses imposed on the catheter by, e.g., a patient's movements.

In some embodiments, device 100 may be configured to regulate the temperature of the contents of reservoir 700. For example, device 100 may include miniature, portable chillers and/or heaters for maintaining the requisite temperatures of certain medicaments.

FIG. 1C shows an exploded view of medicament delivery and continuous monitoring device 100, showing the base subassembly, cartridge system 200, clamshell 300, display 101, and a circuit board 107.

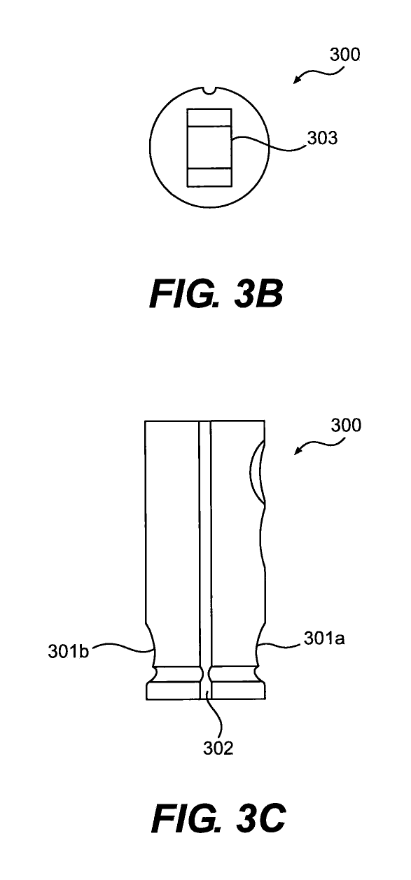

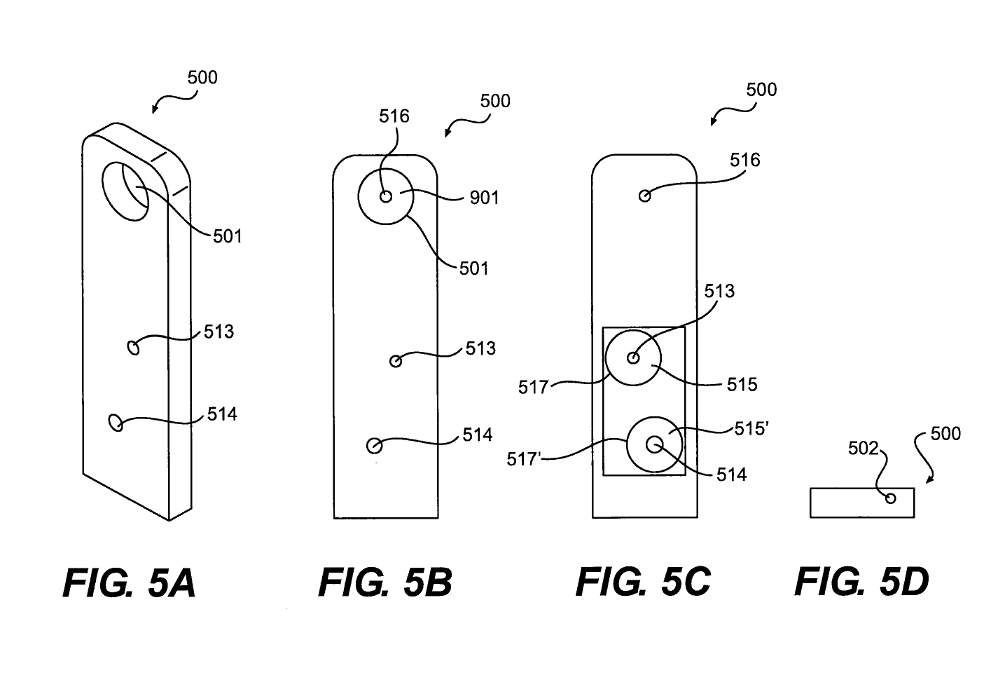

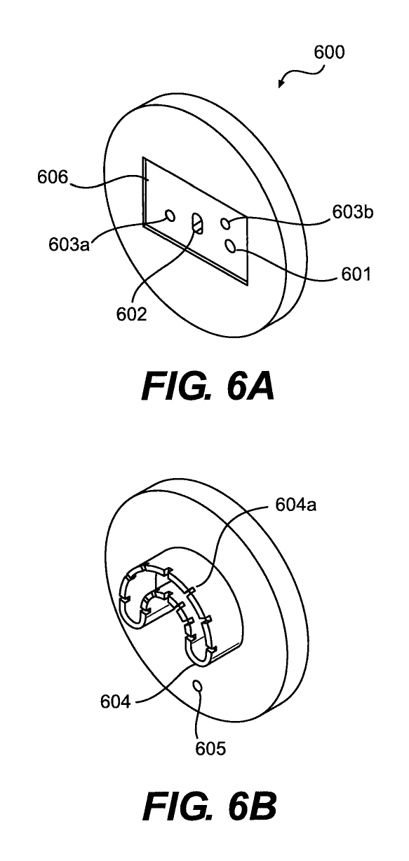

As alluded to above, a signal output from a programmed device 100, or alternatively, depressing a bolus button on device 100, may trigger the micropump (described below in greater detail) to deliver as many bolus doses as required by the patient user. The micropump assembly includes a first pump insert body 400, a second pump insert body 500, a membrane 800 disposed therebetween, a plurality of active valves (not shown) designed and positioned in order to prevent fluid backflow, a plurality of magnets 901 (FIG. 5B) attached to membrane 800, and a plurality of electromagnetic coils 900 (shown in FIG. 1C), which may be made of copper or any other suitable material. First pump insert body 400 and second pump insert body 500 may be secured in clamshell 300, as shown in FIG. 1C. A portion of first and second pump insert bodies 400, 500 may mechanically lock into place, e.g., snap or friction fit, or may be held inside of clamshell 300 through the use of magnets or any other suitable means. Further, a groove 302 (FIG. 3A) on clamshell 300 may align with a corresponding projection on first and second pump insert bodies 400, 500 to facilitate the insertion of first and second pump insert bodies 400, 500 into clamshell 300. Further, the groove and projection mating system may prevent rotation or loosening of the mating parts once secured. One of skill in the art will appreciate that first and second pump insert bodies 400, 500 may include a groove, while clamshell 300 includes a projection, as the groove and projection are interchangeable. A first inlet/outlet member 600 (FIGS. 6A-6B) may provide a fluidic link between reservoir housing 700 and first pump insert body 400 and between first pump insert body 400 and a catheter (not shown), which delivers the medicament to the patient.

Control electronics may be provided on circuit board 107 within device 100 for controlling the micropump assembly, the continuous glucose monitoring module, and display 101. A communication module (not shown) may relay data (wirelessly or through wired connections) between device 100 and a remote controller. An embodiment of the disclosed device may be powered by a one or more batteries (not shown) located in the housing. The batteries may be any suitable batteries known in the art. In some embodiments, the batteries may be single-use batteries, or in other embodiments, the batteries may be batteries that may be selectively rechargeable. In such instances, the batteries may be removed from device 100 and placed into a suitable recharging apparatus until power is fully restored. In even further embodiments, the batteries may be configured to be recharged without requiring removal from within device 100, for example, recharged wirelessly.

As noted above, device 100 may further include a cartridge system 200, as shown in FIG. 2. Cartridge system 200 may include clamshell 300 (shown in FIGS. 3A-3C), a plurality of pump insert bodies 400, 500, inserted into clamshell 300, an inlet/outlet member 600, a reservoir housing 700, a pump membrane 800, a plurality of electromagnetic coils 900 (shown in FIG. 1C), a plurality of electromagnets (not shown), and a plurality of active valves (not shown). Cartridge system 200 may include any suitable shape and/or configuration. For example, as shown in FIGS. 1A-1C, cartridge system 200 may be configured to correspond to a portion of the pen-shaped configuration of device 100.

In some embodiments, cartridge system 200 may be configured for replacement and disposal after a single use. In such an embodiment, reservoir housing 700 of cartridge system 200 may be prefilled by a manufacturer and provided to a user of device 100 for insertion into device 100. In other embodiments, the reservoir of cartridge system 200 may be selectively refillable with one or more desired medicaments. Still further, cartridge system 200 may be configured to provide an indication to a user of the level of contents with the reservoirs of cartridge system 200. For example, cartridge system 200 may have transparent portions that may allow a user to visually inspect the remaining contents through a corresponding transparent portion in reservoir housing 700. In other embodiments, the cartridge housing may have a suitable gauge that communicates the level of contents in reservoir housing 700, as detected by a suitable sensing mechanism placed within reservoir housing 700.

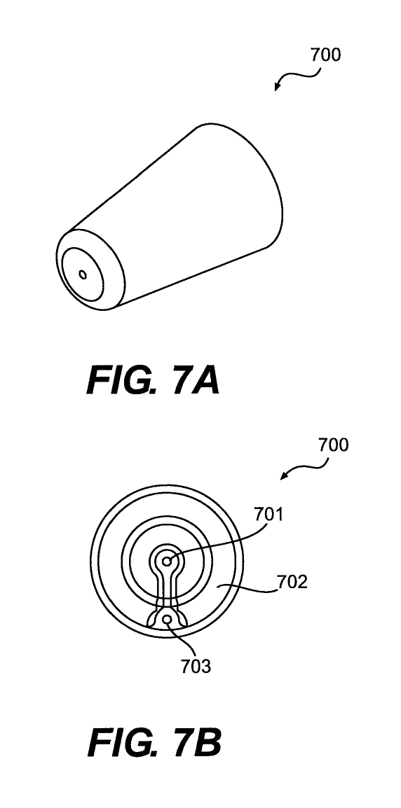

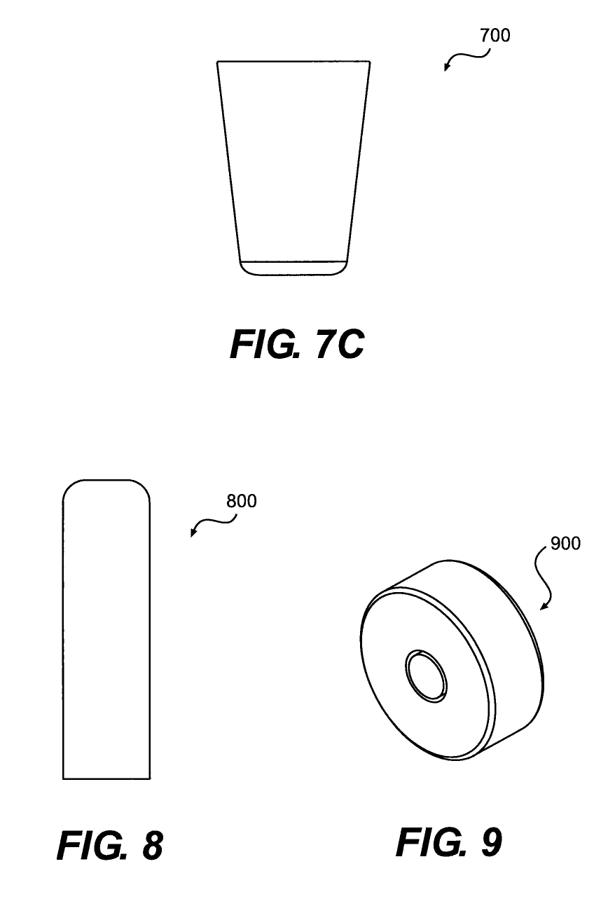

Reservoir housing 700, shown in FIGS. 7A-7C, may include an outlet channel 701 and reservoir cavity 702. While an embodiment containing one reservoir housing 700 and one reservoir cavity 702 is described, it will be appreciated that reservoir housing 700 may include a plurality of cavities 702. In other embodiments, multiple reservoir housings 700 may each include one or more reservoir cavities 702. Whether single or multiple reservoir housings 700 and/or reservoir cavities 702 are used, each may contain one medicament, or optionally, more than one type of medicament. Further, each reservoir cavity 702 could further include multiple trays further subdividing reservoir cavities 702. Reservoir housing 700 may include any suitable container for storing and dispensing suitable medicaments or agents. In one embodiment, reservoir cavity 702 contains a balloon-type reservoir that fills the cavity. Outlet channel 701 may sit in a middle region of reservoir housing 700 and may be configured to channel the medicament away from the device. In one embodiment, the contents of reservoir housing 700 may be delivered to a patient by a suitable pump mechanism, as discussed in greater detail below. In other embodiments, reservoir housing 700 may be a self-emptying reservoir. An alignment hole 703 may be used to prevent rotation of reservoir housing 700 and to help ensure proper alignment with inlet/outlet member 600. Further, one or more walls of each of reservoir housing 700 may be made of an elastic material, which may apply a force to the contents within reservoir housing 700. Reservoir housing 700 can be made of any suitable material, for example, clear acrylic.

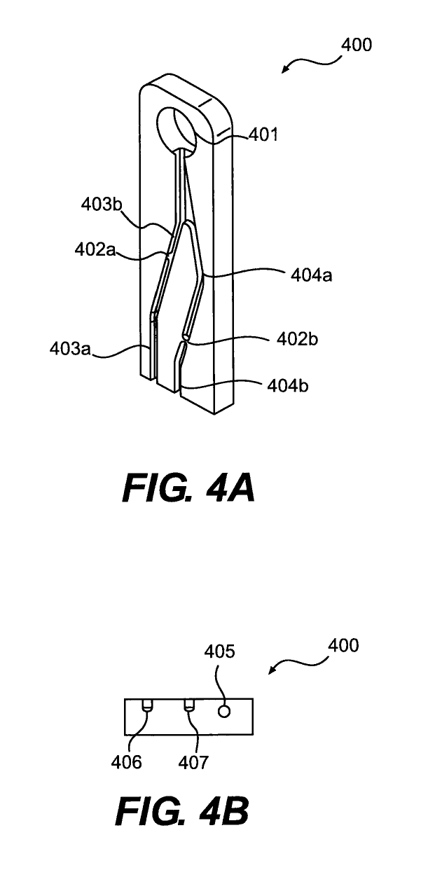

Turning back to FIGS. 3A-3C, clamshell 300 having a plurality of openings 301a, 301b, 301c, and 301d, which are configured to house a plurality of electromagnetic coils 900 is shown. Clamshell 300 may also include an aperture 303 (FIG. 3B) designed to receive and house pump bodies 400, 500. Openings 301a, 301b, 301c, and 301d, may be designed to line up with pump insert body 400 (FIG. 4A) where magnets and valves capable of being activated by a magnetic field are located. Clamshell 300 may further include flanges or any other suitable geometric features designed to facilitate a smooth and preferable insertion of cartridge system 200 into clamshell 300. For instance, clamshell 300 may further include a guide channel 302, which may include a groove that is be configured to correspond with a projection located on cartridge system 200 to facilitate insertion or to help prevent rotation or loosening once cartridge system 200 and clamshell 300 are inserted in the device. This may also help to maintain alignment with the electrical connections to the electric controller. One of skill in the art will appreciate that cartridge system 200 may include a groove, while clamshell 300 includes a projection, as the groove and projection are interchangeable. Clamshell 300 may be made of polyvinyl chloride (PVC) or any suitable material known in the art.

Focusing now on FIGS. 4A-4B, a first pump insert body 400, having one or more magnets (not shown) in one or more magnet housings 401, valve housings 402a, 402b, and a plurality of fluid channels 403, 404, is shown. Initially, those of ordinary skill in the art will recognize that although first pump insert body 400 includes a substantially planar configuration, any suitable configuration may be used within the principles of the present disclosure. First pump insert body 400 may include an elongate member having a first curved end by magnet housing 401, and a second, straight end disposed opposite the curved end. The second, straight end may include a length larger than a diameter of the first curved end. The fluid receiving and discharge openings at the end of channels 403, 404 may be disposed adjacent the second, straight end.

First pump insert body 400 may include a plurality of fluid channels 403, 404. Input channel 403 and output channel 404 may be subdivided further into a plurality of sections, for instance, two input channels 403a, 403b, and two output channels 404a, 404b, as shown in FIG. 4A. However, those of ordinary skill in the art will recognize that any suitable number of fluid channels may be provided. Input channels 403a, 403b and output channels 404a and 404b may be separated by valve housings 402a and 402b, respectively. Valve housings 402a and 402b may house one-way valves to prevent backflow of fluid through fluid channels 403 and 404, as discussed further below. The valve membranes may be active valves magnetically operated and integrated into the membrane housing to control the opening and closing of the output flow. Feedback control may allow for automatic opening or closing of the valve and dispersion of the medicament associated with the reservoir and the corresponding valve. Alternatively, the valves may be self-actuating valves configured to open or close based on changes in fluid pressure as fluid is discharged from reservoir housing 700.

Fluid may flow up from reservoir cavity 702 in reservoir housing 700, into input channel 403a through receiving opening 406 (FIG. 4B). Fluid may then pass through a valve in valve housing 402a, and into the second part of the inlet channel, 403b. The valve in 402a may be configured so as to prevent fluid from flowing back into 403a, thus forcing the fluid to continue into outlet channel 404a. From there, the fluid will travel through valve housing 402b, holding a valve configured to prevent backflow into 404a, and finally into outlet channel 404b and out of discharge opening 407 (FIG. 4B).

Fluid channels 403a, 403b, 404a, 404b may include any suitable shapes and/or configurations. For example, each of fluid channels 403a, 403b, 404a, 404b may include a substantially circular cross-section configuration. Moreover, the cross-sectional configurations of one or more of fluid channels 403a, 403b, 404a, 404b may vary relative to the other of fluid channels 403a, 403b, 404a, 404b. Even further, the cross-sectional configuration of one of fluid channels 403a, 403b, 404a, 404b may vary along its length. In some embodiments, one or more of fluid channels 403a, 403b, 404a, 404b may be provided with a suitable metering mechanism for controlling the flow of fluids through fluid channels 403a, 403b, 404a, 404b.

The plurality of fluid channels 403a, 403b and 404a, 404b may be in fluid communication with the receiving and discharge openings 406, 407, respectively. The plurality of fluid channels 403a, 403b, 404a, 404b may be designed to provide membrane support thereby preventing deformation and reverse flow of fluids. First pump insert body 400 may include an opening 401 to house a magnet (FIG. 4A). Although housing 401 is depicted as disk-shaped, housing 401, or the magnet may include any suitable shape, structure, and configuration.

The fluid is driven by magnets (not shown) in magnet housing 401 through inlet channels 403a, 403b. Inlet valve housing 402a may be synchronized with the upstroke of the magnets in magnet housing 401 to allow flow at only certain times. This may help prevent back-flow of the fluid into reservoir housing 700. The same principle may be used for outlet channels 404a, 404b, with the outlet valve being at valve housing 402b. Inlet channel 403a connects to reservoir 702 in reservoir housing 700. Outlet channel 404b connects to outlet channel 701 in reservoir housing 700. To help align inlet/outlet member 600 with channels 403a, 404b, an alignment hole 405 may be included (FIG. 4B) in first pump insert body 400.

The second pump insert body 500, shown in FIGS. 5A-5C, may be substantially symmetrical in geometry to first pump insert body 400. Second pump insert body 500 may include one or more housings 501 configured to house one or more magnets 901. Housing 501 may be open to the outside environment, for instance, through opening 516, to allow airflow into the housing. This may provide for fluid displacement equal to the amount of flow of medicament through the device. Second pump insert body 500 may also include one or more housings 517, 517′ configured to hold one or more plungers 515, 515′. One or more plungers 515, 515′ may be formed of ferromagnetic stainless steel, or any other suitable material. One or more plungers 515, 515′ may be configured to push on membrane 800, located between first and second pump insert bodies 400 and 500 (FIG. 2). This force is applied from plunger 515 to membrane 800 through openings 513, 514 in second pump insert body 500. The force of plunger 515 may keep membrane 800 securely pressed against inlet/outlet channels 403a, 404b, substantially preventing fluid flow between the channels. When the corresponding electromagnet in housing 301c or 301d of clamshell 300 is energized, plunger 515 is drawn towards the electromagnet, removing the force on membrane 800 and allowing fluid to flow between channels 403a, 404b. Further, second pump insert body 500 may include an alignment hole or projection 502 (FIG. 5D) to help align channels 403, 404 of the first pump insert body 400. First and second pump insert bodies 400, 500 may mate together and may be made of any suitable material, including, but not limited to, clear acrylic.

Cartridge system 200 may further include pump membrane 800, shown in FIG. 8. Membrane 800 may be placed between two disk magnets 901 housed in housings 401, 501 of first and second pump insert bodies 400, 500, respectively (FIG. 2). Disk magnets 901 may include gold-plated neodymium-iron-boron grade N42 magnets. However, any suitable magnets may be used. The volume of fluid medicaments in cartridge system 200 may be related to the diameter of magnets 901 and the stroke length. Magnets 901 may have a preferable diameter of approximately 0.13 inches. The stroke length may be electromagnetically controlled and monitored by a driver feedback system.

Membrane 800 may be configured to act as the primary membrane for pumping action within device 100. Membrane 800 may also provide a sealing edge for the channels of first and second pump insert bodies 400, 500. Membrane 800 may be a biocompatible elastomer membrane, preferably made of Silastic Q7-4840. Further, membrane 800 may have a preferable length of approximately 0.005 inches, a preferable width of approximately 0.32 inches, and a preferable height of approximately 1.13 inches.

Referring now to FIGS. 6A-6C, an inlet/outlet member 600 may facilitate connection between first pump insert body 400 and reservoir housing 700 by, e.g., suitable geometric features (shown more fully in FIG. 2). An inlet channel 601 may be fluidly connected with inlet channel 403a from first pump insert body 400. Inlet channel 601 may also be fluidly connected with a reservoir attachment feature 604. An outlet channel 602 may be fluidly connected with outlet channel 404b from first pump insert body 400. This may connect to outlet channel 701 on reservoir housing 700 (FIG. 7B). A slight bend in the input and output channels 403 and 404 may be introduced to allow for the flow of the fluid to exit along a center region of the device. One or more alignment holes 603a, 603b may be included to help align the inlet and outlet channels properly. Alignment hole or projection 603a may align with a corresponding alignment hole or projection 405 (FIG. 4B) of first pump insert body 400, while alignment hole 603b may align with alignment hole 502 (FIG. 5D) of second pump insert body 500. One of ordinary skill in the art will appreciate that the location of the alignment holes and projections are reversible. Inlet/outlet member 600 may also include a recessed portion 606 corresponding to the shape of the footprint of pump insert bodies 400, 500 to further ensure alignment with pump insert bodies 400, 500. Pump insert bodies 400, 500 may be received within recessed portion 606. To further ensure reservoir housing 700 is aligned, an alignment feature (e.g., hole or projection) 605 may be included opposite the reservoir connection. Inlet/outlet member 600 may be made of polyvinyl chloride (PVC) or any suitable material known in the art.

Turning now to FIG. 9, electromagnetic coils 900 may drive magnets 901 located in magnet housings 401, 501 in first and second pump insert bodies 400, 500, respectively. Electromagnetic coils 900 may also drive the active valves located in first pump insert body 400. Electromagnetic coils 900 may be placed in clamshell 300 and may preferably be made of copper wire or other suitable material.

Device 100 may receive instructions for a bolus event by one of a number of ways. For example, a user may depress button 102c to begin the bolus event. Alternatively, the bolus event may be triggered by a preprogrammed algorithm within the electronics of device 100 or a handheld controller or a Bluetooth device (not shown). Moreover, the bolus event may be selectively triggered by a user via the handheld controller or a Bluetooth device. Alternatively, the bolus event may be triggered by an application programmed to initiate bolus events when required, or by a healthcare provider monitoring the patient through an application and capable of triggering the bolus event remotely.

Once the bolus event is triggered, the electronics within device 100 may cause one or more batteries to power one or more electromagnetic coils 900. Once energized, electromagnetic coils 900 may attract one or both of magnets 901, which may in turn distort membrane 800, resulting in a volumetric change within the pumping chamber. This may allow medicament to flow from reservoir housing 700, through fluid channels 403a, 403b, 404a, 404b, and into a catheter (not shown), delivering a bolus dose to the patient.

As alluded to above, device 100 may be in communication with any suitable infusion set or any suitable mechanism for delivering medicaments to a patient's body. Further, device 100 may be operably coupled with any suitable glucose sensor, for instance, a continuous glucose sensor, configured to monitor the blood glucose of a patient. The sensor may include any suitable housing containing relevant electronics. Further, the sensor may sense a patient's blood glucose by any known sensing technologies, including, but not limited to, technologies employing chemical and/or optical sensing technologies. Any type of suitable sensor can be used, and indeed, those of ordinary skill in the art will understand that the principles of the present disclosure contemplate using any suitable sensor for monitoring any body parameter, for instance, cholesterol, hormone levels, etc. Nonetheless, and solely for purposes of efficiency, the description herein will be directed to a sensor for continuously monitoring glucose levels within a patient. However, any suitable body parameter may be monitored.

Further, a continuous glucose monitoring sensor attached to device 100 may also be attached to a continuous glucose monitoring cannula, which is the in vivo portion configured to be inserted at, e.g., a 45° angle into the subcutaneous tissue of the diabetic patient. The angle of insertion is merely exemplary, and any suitable angle of insertion, including, e.g., 90°, may be employed within the principles of the present disclosure. The cannula may be configured to penetrate a patient's skin for placement within a patient's blood stream for extended periods of time. Thus, it is contemplated that the cannula may be relatively flexible and include relatively small dimensions. The cannula may also include any suitable coating desired. For example, the cannula may be coated with anticoagulation and/or antibiotic agents.

In some embodiments, device 100 may be operatively coupled to a continuous glucose monitoring sensor either physically, e.g., via a catheter or cable, or through a wireless or Bluetooth connection. Any physical connecting mechanisms may be hidden in or beneath clothing so as to help promote the discreetness of device 100. Physical connections between device 100 and a continuous glucose monitoring cannula may be similarly arranged. Any suitable mechanism of continuously monitoring a body parameter may be used within the principles of the present disclosure. For example, a fully implantable sensor module (not shown) may be implanted within a patient's body for extended periods of time. Such a module may be self-contained and self-sustaining. For example, the implantable module may include electronics, algorithms, and other components necessary to detecting the body-parameter (e.g., glucose) and communicating it to system 100 wirelessly. For example, the implantable module may be a small chip or self-contained electronics module. To that end, the implantable module may include a long-lasting power source, or a power source that may recharged wirelessly by, e.g., remote power conduction or induction.

FIGS. 10A-10B illustrate a medicament delivery and glucose monitoring device 1000 in accordance with another exemplary embodiment of the disclosure. The embodiment depicted in FIGS. 10A-10B may share similar components and may function in similar manners as the exemplary embodiments depicted in FIGS. 1A-9. Accordingly, any of the features, components, or details of the embodiment discussed above may also apply to the embodiment of FIGS. 10A-10B, and vice versa.

In this embodiment, device 1000 may include an upper housing 2000 and a lower housing 2100. Device 1000 may also include a cartridge housing 2200, a battery cover 2300, a pen clip 2400, a screen 3000, a bolus button 3100, a plurality of navigation buttons 3200a, 3200b, a circuit board (not shown), a clamshell (not shown), a plurality of electromagnetic coils (not shown), a male modular contact (not shown), a plurality of pump insert bodies (not shown), a flexible pump membrane (not shown), a plurality of permanent magnets (not shown), a plurality of check valves (not shown), a plurality of O-ring seals (not shown), an inlet/outlet member 6600, a female modular contact (not shown), a modular contact circuit board (not shown), a reservoir (not shown), a glucose test strip 8000, and a battery (not shown).

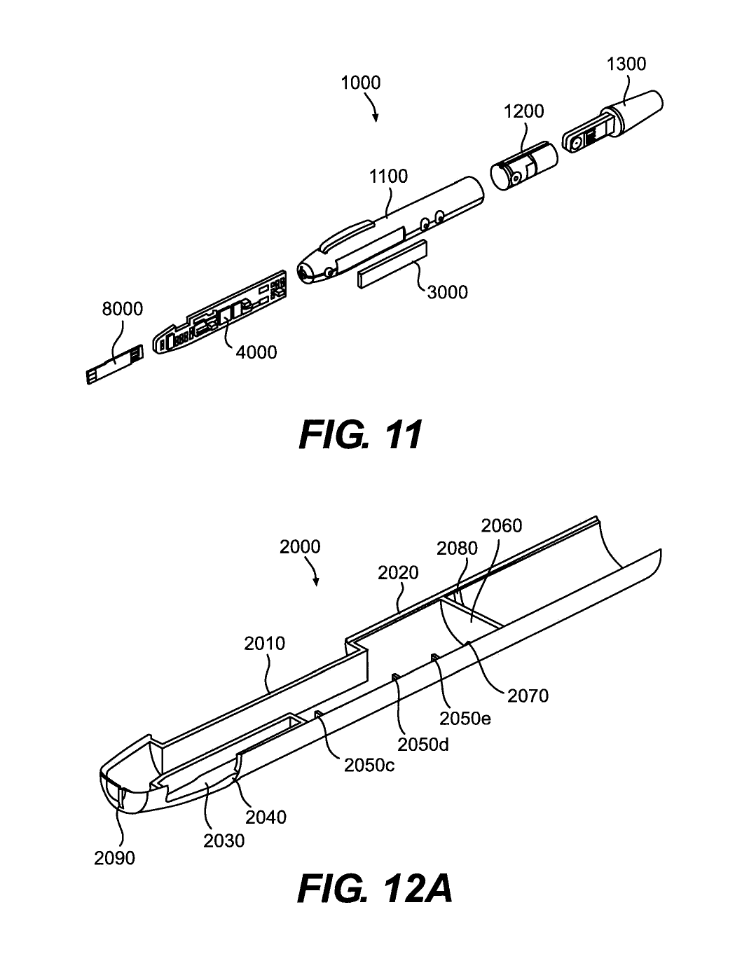

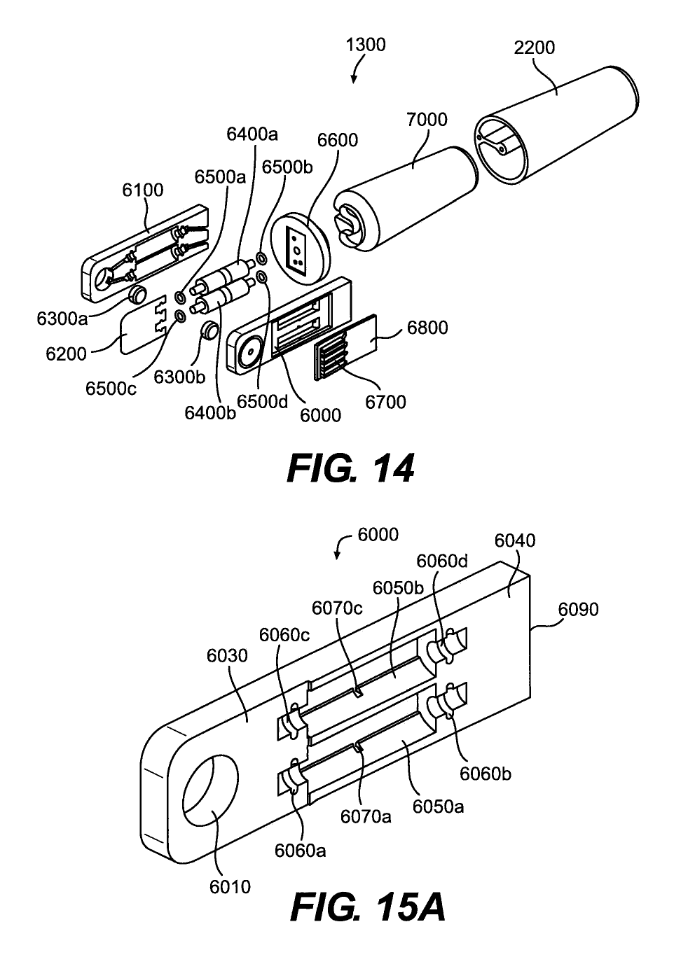

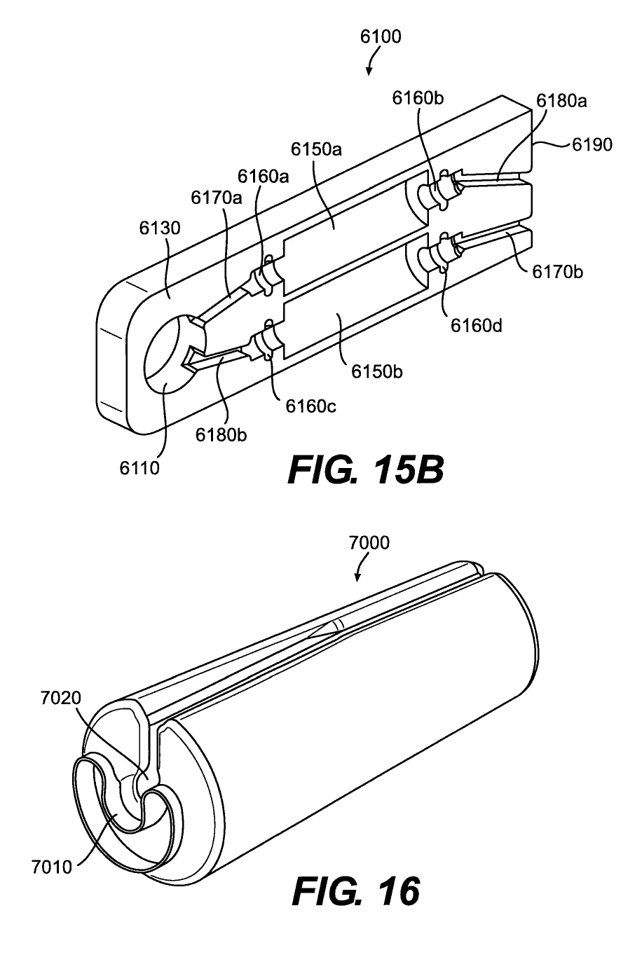

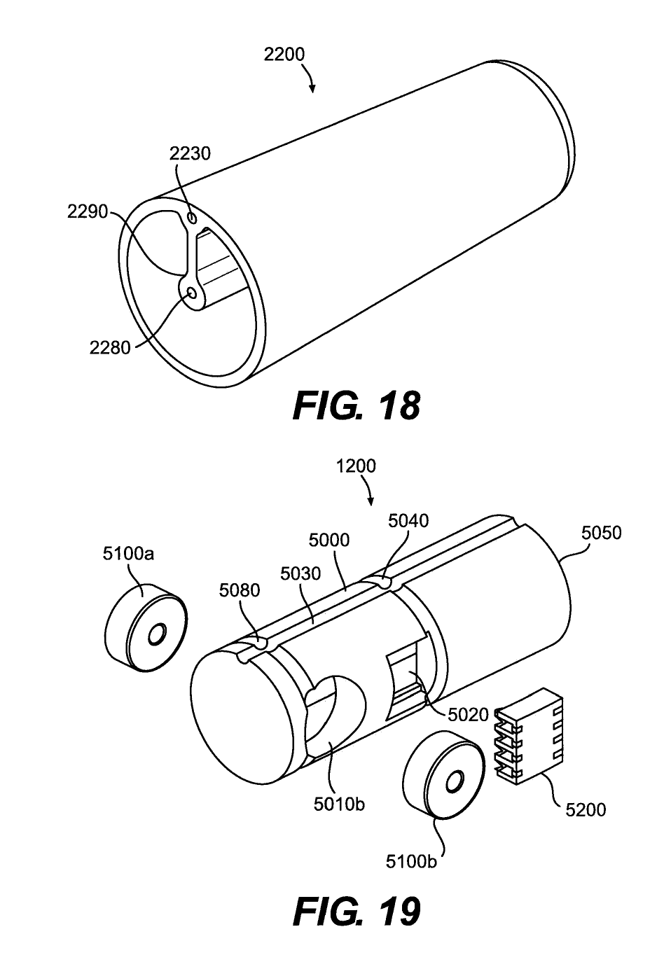

FIG. 11 shows an exploded view of medicament delivery and glucose monitoring device 1000, depicting a housing subassembly 1100, a clamshell subassembly 1200, a cartridge system 1300, a screen 3000, a circuit board 4000 and a glucose test strip 8000. Depressing bolus button 3100 may trigger the micropump that delivers as many bolus doses as required by the patient. The micropump assembly may further include a first pump insert body 6000 (FIG. 15A) upon which a subassembly composed of a female modular contact 6700 soldered on a modular contact circuit board 6800 (FIGS. 13-14) may be mounted, a second pump insert body 6100 (FIG. 15B), a flexible pump membrane 6200 (FIG. 14) disposed therebetween, two check valves 6400a, 6400b (FIG. 14) positioned and controlled to prevent fluid backflow, four O-ring seals 6500a-6500d (FIG. 14) to isolate check valve inlets and outlets from one another, two disk magnets 6300a, 6300b (FIG. 14) attached to flexible membrane 6200 and two electromagnetic coils 5100a, 5100b (FIG. 19). First and second pump insert bodies 6000, 6100 may be secured inside a clamshell 5000 (FIGS. 11, 19) in which two electromagnetic coils 5100a, 5100b and a male modular contact 5200 may be located. Inlet/outlet member 6600 (FIGS. 13-14 and 17A-17B) may provide a fluid connection between reservoir housing 7000 (FIG. 16) and first and second pump insert bodies 6000, 6100 and between first and second pump insert bodies 6000, 6100 and cartridge housing 2200 (FIG. 18) showing an outlet fluidic channel. Circuit board 4000 may include control electronics. The electronics may be configured to communicate with other devices, for instance, a handheld controller. The electronics may also be configured to control the micropump assembly and screen 3000 in accordance with user-directed input signals from buttons 3100, 3200a, 3200b. In a preferred embodiment, device 1000 may be powered by a dry cell battery (not shown) that may be removed from housing subassembly 1100 when necessary.

Referring to FIGS. 12A-12B, upper housing 2000 and lower housing 2100 are shown. Upper housing 2000 may include an opening 2010 which, when mated with opening 2110 of lower housing 2100, may be designed to house screen 3000. An inner groove 2020 in the upper housing may be designed to align with an inner lip 2120 of lower housing 2100. An opening 2030 in upper housing 2000 may be included and configured to house a battery when mated with opening 2130 of lower housing 2100, and battery cover 2300 (FIGS. 10A-11) may be designed to pivot around a pair of cavities 2040, 2140 in upper and lower housings 2000, 2100 so it can open and close battery openings 2030, 2130. A plurality of ribs 2050a-2050e in upper housing 2000 may be configured to pair with a plurality of ribs 2150a-2150e in lower housing 2100 to secure circuit board 4000 (FIG. 11) within the housing. Walls 2060 and 2160 in upper housing 2000 and lower housing 2100, respectively, may be inserted in order to separate the electronic portion of device 1000 from the micropump. An aperture 2070 may be included in upper housing 2000 to allow wires to pass from electromagnetic coils 5100a, 5100b and male modular contact 5200 of clamshell subassembly 1200 (FIGS. 11, 19) to circuit board 4000. Circular ribs 2080, 2180 in upper housing 2000 and lower housing 2100, respectively, may be designed to align with a groove 5080 of clamshell 5000 (FIG. 19), therefore prevent it from sliding off the housing. Slots 2090, 2190 in upper housing 2000 and lower housing 2100 may be designed to receive and/or store one or more glucose test strips 8000, for instance. Upper housing 2000 and lower housing 2100 may be formed of any suitable material, for example, Acrylonitrile butadiene styrene (ABS).

In one exemplary embodiment, device 1000 may prompt a user to take a test strip reading using one of the stored glucose test strips 8000 contained in slots 2090, 2190 in upper housing 2000 and lower housing 2100. This prompt may come in any form, for instance a vibration, a visual prompt, for example, on screen 3000, or an auditory prompt. The user may remove a test strip 8000 from device 1000, place a sample of blood on test strip 8000, and insert it into a suitable sensor module (not shown) provided on either device 1000, a controller, or a remote sensor physically or wirelessly connected to device 1000 and/or the controller. In one embodiment, the controller could include a smart phone having an application configured to facilitate use of device 1000, for instance, by storing data, initiating prompts, communicating data (e.g., to healthcare providers), or reading results. Regardless of where the sensor module is located, the device may then calculate the user's blood glucose level based on the blood sample. The results of the test strip reading may be displayed on screen 3000 of device 1000 and/or on a screen (not shown) of an external controller or sensor, for instance, a smart phone. An algorithm may then compare the results of the test strip reading to a desired glucose level. This algorithm could be programmed either into device 1000 or a controller, for instance, an application on a smart phone. Further, device 1000 or the controller could be operably connected, either physically or wirelessly, to a continuous glucose monitor. The results from the test strip reading may also be compared with the readings derived from the continuous glucose monitor or used to as a form of quality control.

If the test strip reading is within range of the desired glucose level, then no action may be taken. If the reading is too high, however, device 1000 may be triggered to deliver a bolus dose. If, however, the test strip reading indicates that the user's blood glucose level is relatively low, device 1000 may be automatically adjusted to reduce the delivery rate of bolus or basal doses. Regardless of the action taken, the controller or device 1000 may prompt the user to take another test strip reading at the next predetermined interval, or to take another test strip reading right away if the controller or device 1000 sense that an error may have occurred. Alternatively, rather than automatically triggering a bolus dose, device 1000 or a controller may prompt the user to initiate the bolus dose. The bolus dose may be triggered in a manner like the one described above.

FIGS. 13-14 show a disposable cartridge system 1300. Cartridge system 1300 may further include a cartridge housing 2200, first and second pump insert bodies 6000, 6100, flexible membrane 6200, disk magnets 6300a, 6300b, check valves 6400a, 6400b, O-ring seals 6500a-6500d, inlet/outlet member 6600, female modular contact 6700, a modular contact circuit board 6800 and reservoir housing 7000.

Pump membrane 6200 may be a biocompatible elastomer membrane, preferably made of Silastic Q7-4840 and of preferable thickness of approximately 0.005 inches. Disk magnets 6300a, 6300b may be attached to flexible membrane 6200 through any suitable chemical or mechanical means, e.g., glue or adhesives. Disk magnets 6300a, 6300b may include gold-plated neodymium-iron-boron grade N42 magnets. However, any suitable magnets may be used. The volume of fluid medicaments in cartridge system 1300 may be related to the diameter of the magnets and the stroke length. Preferably, disk magnets 6300a, 6300b may have a diameter of approximately 0.13 inches. The stroke length may be electromagnetically controlled and monitored by a driver feedback system.

During typical pump operation, electromagnets 5100a, 5100b (FIG. 19) are energized in alternating polarity to drive magnets 6300a, 6300b, and consequently membrane 6200, in a reciprocating motion. The result of this motion is a volumetric change within pump chamber 6010, 6110. In one stroke direction, a volumetric increase occurs within the pump chamber. This, combined with the timed opening of inlet valve 6400b (with outlet valve 6400a closed), results in a net flow of fluid from reservoir 7000 into the pump chamber. In the opposite stroke direction, a volumetric decrease occurs within the pump chamber. This, combined with the timed opening of outlet valve 6400a (with inlet valve 6400b closed), results in a net flow out of the pump. In another embodiment of this device, the valve operation of outlet valve 6400a and inlet valve 6400b is replaced by valves contained in housings 501 and 501′, which are driven by electromagnets contained within openings 301c and 301d (FIG. 3A).

Focusing now on FIGS. 15A-15B, first pump insert body 6000 and second pump insert body 6100, are shown. First pump insert body 6000 may include an opening 6010 to house disk magnet 6300b. Opening 6010 may be open to the outside environment on one or more sides, allowing air to communicate with the pump mechanism and the housing. This configuration may provide for fluid displacement equal to the amount of flow of medicament through the device, and may be included to allow the pump to be actuated on only one side. This feature may be useful, for instance, in embodiments of device 1000 having multiple reservoir cavities 7020. Each cavity may be filled with a different medicament, and by being able to selectively actuate one side of the pump at a time, device 1000 may be configured to alternatively dispense one type of medicament from one reservoir 7020, or multiple types of medicaments from multiple reservoirs 7020.

Flexible pump membrane 6200 may be located between mating surface 603 of first pump insert body 6000 and mating surface 6130 of second pump insert body 6100. Mating surface 6040 of first pump insert body 6000 may be attached to a portion of mating surface 6130 of second pump insert body 6100 that is not in contact with flexible membrane 6200. Mating surfaces 6040 and 6130 may be attached through any suitable chemical or mechanical means, e.g., glue or adhesives.

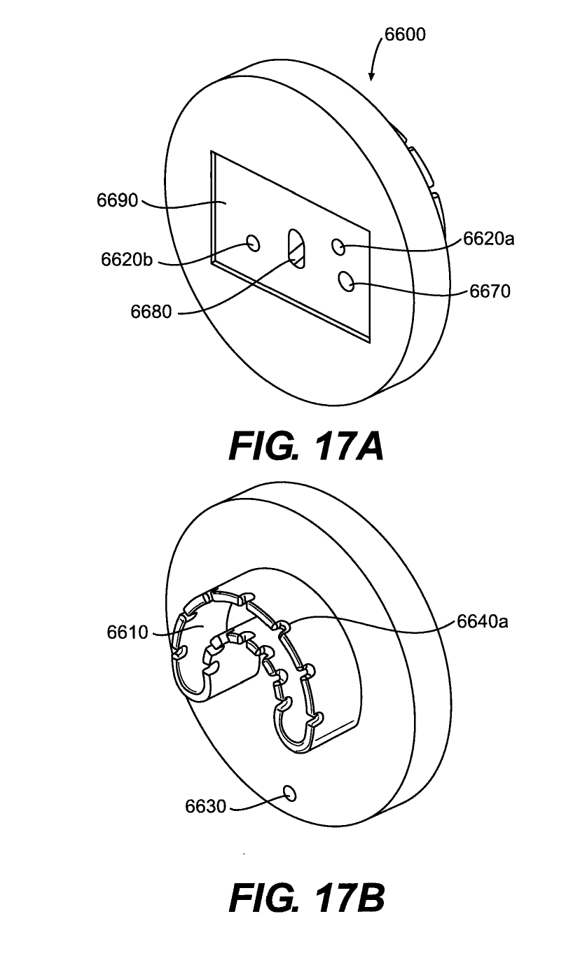

Cavities 6050a and 6150a may be configured to house check valve 6400a, and cavities 6050b and 6150b may be configured to house check valve 6400b. Four O-ring seals 6500a-6500d may be housed in toric cavities 6060a-6060d of first pump insert body 6000 and 6160a-6160d of second pump insert body. While four O-ring seals are depicted here, any suitable number or configuration of O-ring seals may be used. Second pump insert body 6100 may further include a plurality of channels 6170a, 6170b, 6180a, 6180b. Channels 6170a, 6170b may be located upstream from check valves 6400a, 6400b. Channels 6170a, 6170b may be fluidly connected to pump chamber 6010 (FIG. 15A) and reservoir opening 7010 of reservoir housing 7000 (FIG. 16) through aperture 6670 (FIG. 17A) and male part 6610 of inlet/outlet member 6600 (FIG. 17B). Channels 6180a, 6180b may be located downstream from check valves 6400a, 6400b. Channels 6180a, 6180b (FIG. 15B) may be fluidly connected with outlet 2280 of cartridge housing 2200 (FIG. 18) through aperture 6680 of inlet/outlet member 6600 (FIG. 17A), and with pump chamber 6010 (FIG. 15A), respectively. Female modular contact 6700 and its circuit board 6800 (FIG. 14) may be mounted on first pump insert body 6000, and apertures 6070a-6070d and may allow electrical contact between check valves 6400a, 6400b and female modular contact 6700. Female modular connector 6700 may conduct on/off signals to active valves 6400a and 6400b. Alignment holes in first pump insert body 6000 and second pump insert body 6100 may align with alignment holes 6620a, 6620b of inlet/outlet member 6600 to help align pump channels 6170b, 6180a with inlet aperture 6670 and outlet apertures 6680 of inlet/outlet member 6600. Mating surface 6090 of first pump insert body 6000 and mating surface 6190 of second pump insert body 6100 may also be attached to surface 6690 of the inlet/outlet member through any suitable chemical or mechanical means, e.g., glue or adhesives. First pump insert body 6000 and second pump insert body 6100 are preferably made of clear acrylic. However, any suitable material may be used.

Referring now to FIG. 16, reservoir housing 7000 having a hollow, reservoir cavity 7020 is shown. While an embodiment containing one reservoir housing 7000 and one reservoir cavity 7020 is described, it will be appreciated that reservoir housing 7000 may include a plurality of cavities 7020. In other embodiments, multiple reservoir housings 7000 may each include one or more reservoir cavities 7020. Whether single or multiple reservoir housings 7000 and/or reservoir cavities 7020 are used, each may contain one medicament, or optionally, more than one type of medicament. Further, each reservoir cavity 7020 could further include multiple trays further subdividing reservoir cavities 7020. In an exemplary embodiment, reservoir housing 7000 may include a reservoir cavity 7020 located in a middle region and configured to allow outlet 2280 of cartridge housing 2200 to pass through it (FIG. 18), thus connecting fluid in reservoir cavity 7020 to the pump system. A first inlet/outlet member 6600 (FIGS. 17A-17B) may provide a fluidic link between reservoir housing 7000, the pump system, and a catheter (not shown), which delivers the medicament to the patient. Accordingly, outlet channel 2280 may be configured to deliver medicament from an outlet channel in the pump system to the patient via a fluid connection with a catheter (not shown).

Reservoir housing 7000 may be made of elastomers and preferably made by liquid injection molding of Silastic Q7-4840 or transfer molding of Medical Grade Polyisoprene. However, any suitable method of manufacture and material may be used. A polymer reservoir may allow better use of the interior volume available within the cartridge body, and the collapsible nature of the material may allow for more innovative methods for withdrawing the liquid contents. Reservoir housing 7000 preferably contains a volume of medicament of approximately 1500 or more units.

Referring now to FIGS. 17A-17B, a cylindrical inlet/outlet member 660 including inlet aperture 6670 and outlet 6680, is shown. Alignment holes or projections 6620a, 6620b may be included to help align inlet/outlet member 6600 with first and second pump insert bodies 6000, 6100. Further, an alignment hole or projection 6630 may allow inlet/outlet member 6600 to align with cartridge housing 2200 through alignment hole or projection 2230 (FIG. 18). Inlet/outlet member 6600 is preferably made of clear acrylic. However, any suitable material may be used.

Referring to FIG. 18, cartridge housing 2200 may be configured to house reservoir housing 700. Reservoir housing 7000 may be secured in cartridge housing 2200, as shown in FIG. 14. A portion or all of reservoir housing 7000 may mechanically lock into place, e.g., snap or friction fit, or may be held inside of cartridge housing 2200 through the use of magnets or any other suitable means. Further, a large, rib-like projection 2290 (FIG. 18) on cartridge housing 2200 may align with a corresponding groove extending the length of reservoir housing 7000 to facilitate the insertion of reservoir housing 7000 into carriage housing 2200. Further, the groove and projection mating system may prevent rotation or loosening of the mating parts once secured. This may help to secure outlet channel 2280 in place and maintain fluid flow between reservoir cavity 7020, cartridge housing 2200, inlet/outlet member 6600, and the pump system. As mentioned above, first inlet/outlet member 6600 (FIGS. 6A-6B) may provide a fluidic link between reservoir housing 7000, the pump system, and a catheter (not shown), which delivers the medicament to the patient. Outlet channel 2280 may be configured to deliver medicament from an outlet channel in the pump system to the patient via a fluid connection with a catheter (not shown). One of skill in the art will appreciate that the corresponding groove and projection features are interchangeable. Cartridge housing 2200 is preferably made of Acrylonitrile butadiene styrene (ABS) or equivalents. However, any suitable material may be used.

Referring now to FIG. 19, a clamshell subassembly 1200 composed of clamshell 5000, electromagnetic coils 5100a, 5100b, and male modular contact 5200, is shown. Electromagnetic coils 5100a, 5100b may be housed in openings 5010a, 5010b of clamshell 5000, and male modular contact 5200 may be housed in opening 5020 of clamshell 5000. An aperture 5050 may be configured to house first pump insert body 6000 and second pump insert body 6100, as depicted in the exploded view in FIG. 11. Electrical contact may be made between electromagnetic coils 5100a, 5100b and circuit board 4000, and between check valves 6400a, 6400b and circuit board 4000 when cartridge system 1300 (FIG. 13) is inserted into clamshell subassembly 1200 as a result of the electrical contact between male and female modular contacts 5200, 5700. Grooves 5030, 5080 of clamshell 5000 may be designed to help prevent rotation and translation of clamshell 5000 with respect to upper housing 2000 and lower housing 2100. Those grooves, along with groove 5040, may also make electrical contact possible by allowing wires to pass through them. Clamshell 5000 may be made of polyvinyl chloride (PVC) or any suitable material known in the art.

Various components of the embodiments described herein may have one or more of the exemplary parameters in Table 1 below.

TABLE 1
Exemplary Parameters

| | |
|---|---|
| Lower housing | |
| Overall dimensions | 4.26″ (length) × 0.50″ to 0.68″ (diameter) |
| Material | ABS |
| Upper housing | |
| Overall dimensions | 4.26″ (length) × 0.50″ to 0.68″ (diameter) |
| Material | ABS |
| Battery flap | |
| Overall dimensions | 0.50″ (length) × 0.19 (width) × 0.05 (thickness) |
| Material | ABS |
| Pen clip | |
| Overall dimensions | 1.18″ (length) × 0.18 (width) × 0.21 (thickness) |
| Material | ABS |
| Navigation button | |
| Overall dimensions | 0.25″ (diameter) × 0.03″ (thickness) |
| Material | ABS |
| Bolus button | |
| Overall dimensions | 0.14″ (diameter) × 0.05″ (thickness) |
| Material | ABS |
| Cartridge housing | |
| Overall dimensions | 0.80″ (length) × 0.40 to 0.57″ (diameter) |
| Material | ABS |
| Screen | |
| Overall dimensions | 1.50″ (length) × 0.25″ (height) × 0.08 (thickness) |
| Material | Liquid Crystal Display or Light Emitting Diode |
| Clamshell | |
| Overall dimensions | 0.50″ (diameter) × 1.18″ (length) |
| Material | PVC |
| Electromagnetic coil | |
| Overall dimensions | 0.250″ (diameter) × 0.100″ (height) |
| Material | Copper |
| Male modular | |
| contact | |
| Overall dimensions | 0.20″ (length) × 0.22″ (width) × 0.08″ (thickness) |
| Material | Glass reinforced thermoplastic, copper alloy |
| Female modular | |
| contact | |
| Overall dimensions | 0.20″ (length) × 0.22″ (width) × 0.04″ (thickness) |
| Material | Glass reinforced thermoplastic, copper alloy |
| Modular contact | |
| circuit board | |
| Overall dimensions | 0.36″ (length) × 0.28″ (width) × 0.02″ (thickness) |
| Material | Epoxy resin pre-preg |
| Pump insert body | |
| Overall dimensions | 1.13″ (length) × 0.32″ (width) × 0.09″ (thickness) |
| Material | Clear acrylic |
| Inlet/outlet member | |
| Overall dimensions | 0.57″ (diameter) × 0.22″ (thickness) |
| Material | Clear acrylic |
| Check valve | |
| Overall dimensions | 0.12″ (diameter) × 0.59″ (length) |
| Material | Stainless steel, polymer, ceramic, epoxy |
| O-ring seal | |
| Overall dimensions | 0.09″ (diameter) × 0.02″ (width) |
| Material | Silicone |
| Flexible pump | |
| membrane | |
| Overall dimensions | 0.44″ (length) × 0.32″ (width) × 0.005″ (thickness) |
| Material | Silastic Q7-4840 |
| Disk magnet | |
| Overall dimensions | 0.13″ (diameter) × 0.06″ (height) |
| Material | Neodymium-iron-boron grade N42 magnets, |
| | gold plated NdFeB |
| Reservoir | |
| Overall dimensions | 0.75″ (length) × 0.31 to 0.48″ (diameter) |
| Material | Silastic Q7-4840 or Medical Grade Polyisoprene |

Further, In some embodiments, it is contemplated may include additional optional features. Such features may include, but are not limited to, circuitry relating to fitness and/or a user's lifestyle. For example, system may include an integrated pedometer, a global positioning system (GPS), a music player, and so forth.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

What is claimed is:

1. A medical device for delivering a medicament to a body of a user, comprising:
an elongate housing;
a reservoir configured to contain the medicament;
a delivery mechanism for channeling the medicament from the reservoir to the user;
a pumping mechanism for pumping the medicament from the reservoir, through the delivery mechanism, and to the user, wherein the medical device is configured to deliver a basal dose of the medicament to the user; and
a first sensor for monitoring a parameter of the body.

2. The medical device of claim 1, wherein the elongate housing includes a securing mechanism configured to secure the elongate housing to an article of clothing.

3. The medical device of claim 2, wherein the elongate housing includes dimensions allowing the housing to be disposed substantially within a pocket of the article of clothing.

4. The medical device of claim 1, wherein the reservoir is configured to contain a plurality of medicaments.

5. The medical device of claim 4, wherein the pumping mechanism is configured to deliver a first medicament of the plurality of medicaments at a first rate and to deliver a second medicament of the plurality of medicaments at a second rate, wherein the second rate is different from the first rate.

6. The medical device of claim 1, wherein the pumping mechanism is configured to selectively deliver bolus doses to the user.

7. The medical device of claim 6, wherein the housing includes an actuator configured to allow the user to instruct the medical device to deliver a bolus dose of the medicament.

8. The medical device of claim 1, wherein the elongate housing includes a graphical display screen.

9. The medical device of claim 1, wherein the first sensor is configured to continuously monitor the parameter.

10. The medical device of claim 1, further comprising a device control unit configured to receive data from the first sensor, wherein the device control unit includes a data acquisition system.

11. The medical device of claim 1, wherein the parameter is blood glucose.

12. The medical device of claim 1, wherein the first sensor includes a portion in communication with blood of the user.

13. The medical device of claim 12, wherein the portion is implanted within the body of the user.

14. The medical device of claim 13, wherein the portion is wirelessly coupled to the first sensor.

15. The medical device of claim 1, further comprising a second sensor analyzing a blood sample removed from the body for the parameter.

16. The medical device of claim 1, wherein the reservoir is selectively removable from the housing.

17. The medical device of claim 1, wherein the pumping mechanism includes a magnet and an electromagnetic coil.

18. The medical device of claim 1, wherein the housing includes electronics for controlling the first sensor and the pumping mechanism.

19. The medical device of claim 18, further comprising a handheld controller in wireless communication with the electronics.

20. The medical device of claim 19, wherein the handheld controller includes a second sensor configured to analyze a blood sample removed from the body for the parameter.

21. The medical device of claim 1, wherein the elongate housing has a length in the range of about 3.5 inches to 4.5 inches, and a width in the range of about 0.40 inches to 0.6 inches.

22. The medical device of claim 1, wherein the elongate housing includes a substantially cylindrical configuration.

23. The medical device of claim 1, wherein the elongate housing is substantially pen-shaped.

24. A pump mechanism for delivering a medicament from a reservoir, wherein the reservoir is removably secured to a housing, the pump mechanism comprising:
a first elongate pump insert body having a first opening configured to receive a first magnet;
a second elongate pump insert body having a second opening configured to receive a second magnet;
a flexible membrane disposed in between the first and second elongate pump insert bodies; and
a plurality of electromagnetic coils.

25. The pump mechanism of claim 23, wherein the first and second magnets are disposed in the first and second openings.

26. The pump mechanism of claim 25, wherein the flexible membrane is operably coupled to one of the first and second magnets.

27. The pump mechanism of claim 24, wherein the reservoir is disposed in a housing configured to be secured to an article of clothing.

28. A medical device for delivering a medicament to the body of a user, comprising:
an elongate housing;
a reservoir configured to contain the medicament;
a delivery mechanism for channeling the medicament from the reservoir to the user;
a pumping mechanism for pumping the medicament from the reservoir and through the delivery mechanism to the user, the pumping mechanism comprising:
a first elongate pump insert body having a first opening configured to receive a first magnet;
a second elongate pump insert body having a second opening configured to receive a second magnet;
a flexible membrane disposed in between the first and second elongate pump insert bodies; and
a plurality of electromagnetic coils; and
a first sensor for monitoring a parameter of the body, wherein the medical device is configured to deliver a basal dose of the medicament to the user.
